(19) United States
(12) Patent Application Publication
(10) Pub. No.: US 2017/0328912 A1
(43) Pub. Date: Nov. 16, 2017

(54) GLYCOPOLYMER CAPTURE MATRIX FOR USE WITH SURFACE-ENHANCED RAMAN SPECTROSCOPY DETECTION AND RELATED SYSTEMS AND METHODS

(71) Applicant: REGENTS OF THE UNIVERSITY OF MINNESOTA, MINNEAPOLIS, MN (US)

(72) Inventors: Victoria Marie Szlag, Minneapolis, MN (US); Christy L. Haynes, Minneapolis, MN (US); Theresa Reineke, Vadnais Heights, MN (US); Antonio Campos, Austin, TX (US); Bharat Wagh, Waialua, HI (US)

(21) Appl. No.: 15/591,307

(22) Filed: May 10, 2017

Related U.S. Application Data
(60) Provisional application No. 62/334,203, filed on May 10, 2016.

(51) Int. Cl. G01N 33/68; C09D 133/26; C08F 120/60; G01N 21/65; G01N 33/543; G01N 33/553

(52) U.S. Cl. CPC G01N 33/68; G01N 33/54373; G01N 33/553; C08F 120/60; G01N 21/658; C09D 133/26; G01N 2333/42

(57) ABSTRACT

A method of making a sensor includes subjecting a metal substrate to a solution having glycopolymer chains and attaching the glycopolymer chains to the metal substrate to form a glycopolymer-functionalized metal substrate that can bind a lectin target. A lectin target can be a food allergen or a toxin. The metal substrate can include a plasmonic metal. A method of using the sensor includes incubating a portion of the sensor in a sample fluid and another portion of the sensor in a control fluid. Spectral data sets are generated via Raman Spectroscopy for each portion of the sensor. The presence and concentration of a lectin target in the sample fluid is determined by comparing the spectral data sets.

### CROSS-REFERENCE TO RELATED APPLICATIONS

The present application claims the benefit of U.S. Provisional Patent Application Ser. No. 62/334,203, filed May 10, 2016, which is incorporated herein by reference for all purposes.

### GOVERNMENT FUNDING

This invention was made with government support under DMR-0819885 awarded by the National Science Foundation and N660001-11-1-4179 awarded by the Defense Advanced Research Projects Agency. The government has certain rights in the invention.

### SUMMARY

Aspects of the present disclosure relate to a method of making a sensor. The method includes subjecting a metal substrate to a solution having at least one glycopolymer chain configured to bind to a lectin target. The method further includes attaching the at least one glycopolymer chain to the metal substrate to form a glycopolymer-functionalized metal substrate configured to bind to a lectin target.

The at least one glycopolymer chain may include at least one of N-acetyl-galactosamine, N-acetyl-glucosamine, glucose, galactose, and mannose.

The at least one glycopolymer chain may include a repeat unit of N-acetyl-galactosamine ethyl methacrylamide.

The at least one glycopolymer chain may include at least one of a trithiocarbonate, a thiol, a disulfide, and a dithiocarbonate.

Attaching the at least one glycopolymer chain to the substrate may include chemisorption of the at least one glycopolymer chain to the metal substrate.

The method may further include forming the metal substrate with a plasmonic metal having at least one of gold, copper, and silver.

Aspects of the present disclosure relate to a method of using a sensor. The method includes incubating a first sensor portion in a sample fluid and a second sensor portion in a control fluid. The first and second sensor portions each include a glycopolymer-functionalized metal substrate configured to generate a signal-enhancing electromagnetic field in response to incident light. The method further includes generating, via Raman Spectroscopy, after incubating the first and second sensor portions, a first post-incubation set of spectral data representing the first sensor portion and a second post-incubation set of spectral data representing the second sensor portion. The method also includes determining whether a lectin target is present in the sample fluid in response to comparing the first and second post-incubation sets of spectral data in a shift region. The shift region is dependent on a concentration of the lectin target in the sample fluid.

The method may further include generating, via Raman Spectroscopy, before incubating the first and second sensor portion, a pre-incubation set of spectral data representing the first and second sensor portions. The method may also include determining a potential shift region in response to comparing the pre-incubation set of spectral data to at least one of the first and second post-incubation sets of spectral data.

The shift region may be defined at about 700 cm⁻¹.

The shift region may be defined at about 1280 cm⁻¹, from about 615 cm⁻¹ to about 630 cm⁻¹, at about 380 cm⁻¹, or any combination of two or more thereof.

The shift region may be defined at about 600 cm−1.

The method may further include determining a concentration of the lectin target in the sample fluid in response to comparing peak intensities in the shift region of the first and second sets of spectral data.

Aspects of the present disclosure relate to a sensor. The sensor includes a metal substrate having a plasmonic metal. The sensor further includes at least one glycopolymer chain attached to the metal substrate configured to bind to a lectin target.

The at least one glycopolymer chain may include at least one of N-acetyl-galactosamine, N-acetyl-glucosamine, glucose, galactose, and mannose.

The at least one glycopolymer chain may include a repeat unit of N-acetyl-galactosamine ethyl methacrylamide.

The metal substrate may include at least one of gold, copper, and silver.

The metal substrate may include one of a film of gold over silica nanosphere matrix and a colloidal gold substrate.

The at least one glycopolymer chain may be configured to bind to at least one of a lectin food allergen and a lectin toxin.

The at least one glycopolymer chain may be configured to bind to a ricin B chain.

The at least one glycopolymer chain may be configured to bind to a soybean agglutinin.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Example scheme of sensor fabrication. (I) Silica nanospheres, were drop-coated and self-assembled on a silicon wafer creating a packed sphere mask. (II) 80 nanometers (nm) of gold was deposited onto the nanosphere template, and substrates with a localized surface plasmon resonance (LSPR) λₘₐₓ between 735-780 nm were utilized. (III) The Au film-over-nanospheres (FONs) were immersed in a 1 mM polymer solution for eighteen hours to allow attachment by chemisorption through the trithiocarbonate of the polymer to the gold.

FIG. 2. Comparison of experimental polymer N-acetyl-galactosamine ethyl methacrylamide (pNAGEMA) spectrum (grey, top) and computed NAGEMA spectrum (black, bottom). The experimental spectrum was collected with a 785 nm diode laser with 3 mW power over 30 seconds.

FIG. 3. Spectral differences due to the presence of ricin B-chain (RBC): (A) representative difference spectrum demonstrating changes (a-g) due to RBC, (B) difference spectra of varying concentrations of RBC in the 670-750 cm⁻¹ shift region, (C) quantification of the increased amplitude at 700 cm⁻¹ shift with increasing RBC concentration, including the calculated association constant (Kₐ), and limits of detection (LOD) and quantification (LOQ).

FIG. 4. Detection of RBC in juice: principal component analysis (PCA) plots of raw spectra from RBC (black) and buffer (white) in apple and orange juices are shown in (A) and (C), respectively. Averaged difference spectra of RBC (3 μg/mL) (gray) and buffer (black) in apple juice, and RBC (10 μg/mL) and buffer in orange juice can be seen in (B) and (D), respectively.

FIG. 5. Proton nuclear magnetic resonance (NMR) representation of purified monomer NAGEMA.

FIG. 6. Proton NMR representation of pNAGEMA.

FIG. 7. A comparison of the 20 μg/mL RBC difference spectrum and the 2-mercaptoethanol buffer equivalent of 50 μg/mL difference spectrum. The labeled peaks are those discussed with respect to Table 2.

FIG. 8. Baseline-corrected SERS spectra of different bare FON substrates without polymer incubated in 3 μg/mL RBC or in an equivalent concentration of 2-mercaptoethanol buffer usable as a negative control.

FIG. 9. Baseline-corrected and averaged SERS spectra at ten different spots on each FON substrate for different FON substrates incubated in orange juice with ricin B-chain, orange juice with buffer, apple juice with ricin B-chain, and apple juice with buffer.

FIG. 10. Difference of averaged SERS spectra for different FON substrates comparing post-incubation with pre-incubation for orange juice with ricin B-chain, orange juice with buffer, apple juice with ricin B-chain, and apple juice with buffer.

FIG. 11. Spectral differences due to the presence of soybean agglutinin (SBA): (A) PCA plot of raw spectra from SBA incubation (black), 4-(2-hydroxyethyl)-1-piperazineethanesulfonic acid (HEPES) incubation (white), and pNAGEMA only (gray), (B) averaged spectra of HEPES incubation (white) and SBA incubation (black), and (C) difference spectra post baseline subtraction of SBA incubation (black) and HEPES incubation (white).

### DETAILED DESCRIPTION OF ILLUSTRATIVE EMBODIMENTS

This disclosure describes a novel sensing scheme for lectin detection using surface-enhanced Raman Spectroscopy (SERS) coupled with a glycopolymer capture layer/matrix, as exemplified through the detection of ricin B-chain (RBC) or soybean agglutinin (SBA) in water and liquid food matrices using an N-acetyl-galactosamine glycopolymer. Lectins are saccharide-binding proteins that can be allergenic or toxic to ingest, for example. The sensing scheme's detection limit can be well below that of the predicted oral exposure limit of toxins, such as RBC, or can be used to detect a food allergen, such as SBA. Theoretical predictions of the normal Raman spectrum of the glycomonomer can be used to give insight to polymer-lectin intermolecular interactions.

Ricin, a heterodimeric ribosome-inactivating protein found in castor beans (Ricinus communis), is a highly toxic (oral LD50 of 20 mg/kg) biological agent. Ricin can be used to contaminate food or drink as it is stable in ambient conditions and resistant to heat and denaturants, such as chloride. The mechanism of toxicity involves both the A and B chains of this heterodimeric protein. The B-chain is a lectin, a protein that binds a specific sugar, which controls epithelial cell adhesion and endocytosis via extracellular glycoprotein binding. The A-chain attacks the 28S rRNA of the 60S ribosome subunit, halting protein synthesis and causing apoptosis. When separate, the two ricin subunits are benign, making the RBC lectin an excellent low risk candidate for studying sensing methods.

Soybean agglutinin (SBA) is a tetrameric lectin found in soybeans. Some people with soy allergies demonstrate SBA-specific immunoglobulin E antibodies. SBA has also been shown to cause intestinal inflammation. The 120 kDa protein may have four binding sites capable with binding N-acetyl-galactosamine (GalNAc) and galactose (Gal). The affinity for GalNAc may be approximately 40 stronger than that for Gal.

Raman spectroscopy in the presence of plasmonic nanoscale metal features (SERS) allows one to detect the molecular “finger print” provided by Raman spectroscopy, circumvent the innately poor Raman scattering cross-section, and access ultra-low concentration sensitivity even in complex aqueous matrices.

In SERS, incident light can excite the conduction electrons of the metal features, generating a signal-enhancing electromagnetic (EM) field that typically extends less than 10 nanometers from the substrate surface, though the range may be specific to the plasmonic substrate being used. Conjugating the metal substrates with target-capture agents can allow one to retain a target within this signal-enhancing EM field. Many SERS protein sensors use antibodies or aptamers as capture agents. In the case of proteins with intrinsic affinities, such as lectins, these biological capture agents can be considered redundant, unnecessarily costly to specifically design, and laborious to develop and mass produce. Lectins are proteins that bind a specific sugar.

In contrast, the present disclosure describes a method of making a sensor that includes subjecting a metal substrate to a solution having at least one glycopolymer chain configured to bind to a lectin target; and attaching the at least one glycopolymer chain to the metal substrate to form a glycopolymer-functionalized metal substrate configured to bind to a lectin target. The metal may be a plasmonic metal. The sensor may be used by incubating a first sensor portion with the in a sample fluid. A second sensor portion may be incubated in a control fluid. The first and second sensor portions each include a glycopolymer-functionalized metal substrate configured to generate a signal-enhancing electromagnetic field in response to incident light. After incubating the first and second sensor portions, a first set of spectral data representing the first sensor portion and a second set of spectral data representing the second sensor portion may be generated, for example, via Raman Spectroscopy. Whether the lectin target is present in the sample fluid may be determined by comparing the first and second sets of spectral data in a shift region that is dependent on a concentration of the lectin target. The sensor may include a metal substrate and at least one glycopolymer chain attached to the metal substrate.

In some embodiments, the sensor may be configured to bind a lectin target, which may be a lectin or a component thereof. Such a sensor may be useful in detecting a lectin target that is, for example, a food allergen or a toxin. Non-limiting examples of lectin food allergens include soybean agglutinin, wheat germ agglutinin, and peanut agglutinin. A non-limiting example of a lectin toxin is ricin. A non-limiting example of a lectin toxin component is ricin B chain.

The glycopolymer capture matrix can be more tunable and/or more stable at ambient conditions than an aptamer or antibody. The glycopolymer capture matrix may also have a higher binding affinity with lectins than thiolated carbohydrates. In general, a glycopolymer matrix may facilitate the ease of design, synthesis, and application of a lectin detection sensor.

### Detection Sensitivity

Using the difference in Raman shift due to incubation with RBC, the limit of detection (LOD) and limit of quantification (LOQ) are calculated to be 0.02 μg/mL RBC and 0.08 μg/mL RBC, respectively (for calculation methods, see SI). The limit of quantitation (LOQ) was calculated as 3.3*LOD.

According to this disclosure, RBC detection was sufficiently sensitive for applications related to bio-terror prevention in food matrices. The quantitative concentration dependence seen at 700 cm⁻¹ shift band in simple media has an LOQ (80 ng/mL) well below the toxic limit for oral exposure.

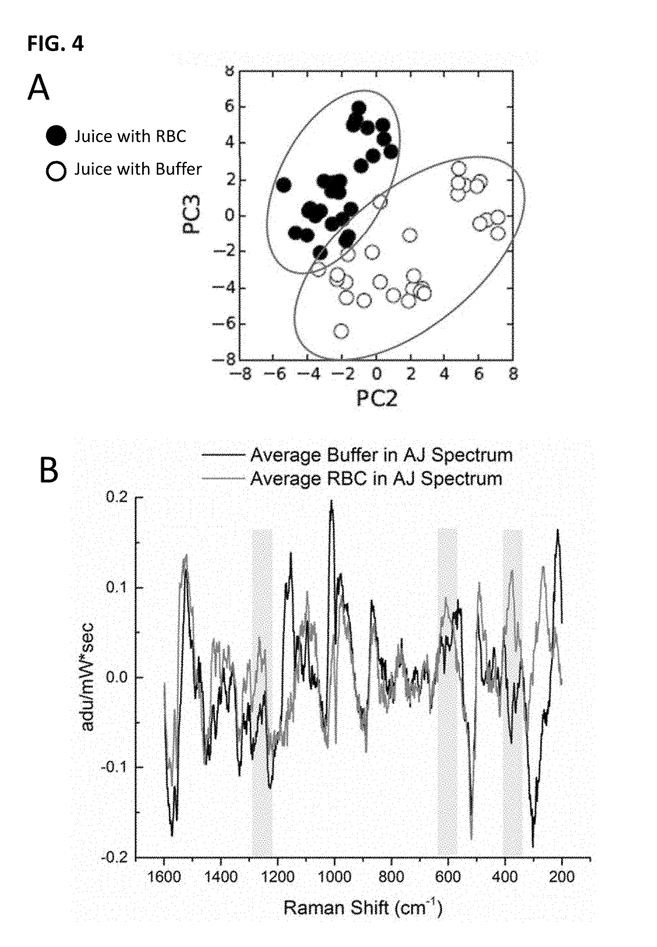

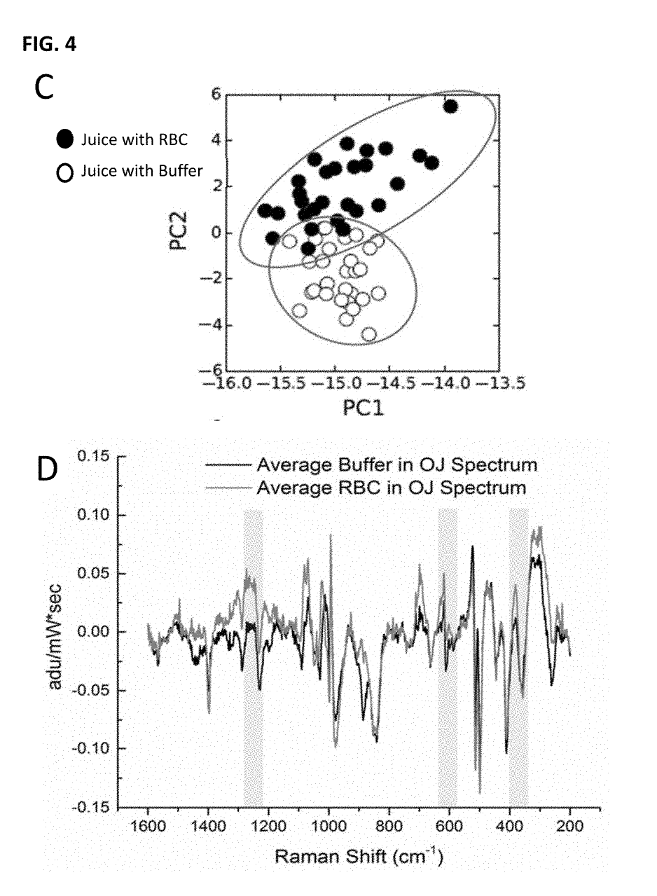

As seen in FIG. 4, the principle component analysis (PCA) of the control and spiked juice conditions (FIGS. 4A and 4C) reveal that there is variance in the spectra that correlates with the presence of RBC.

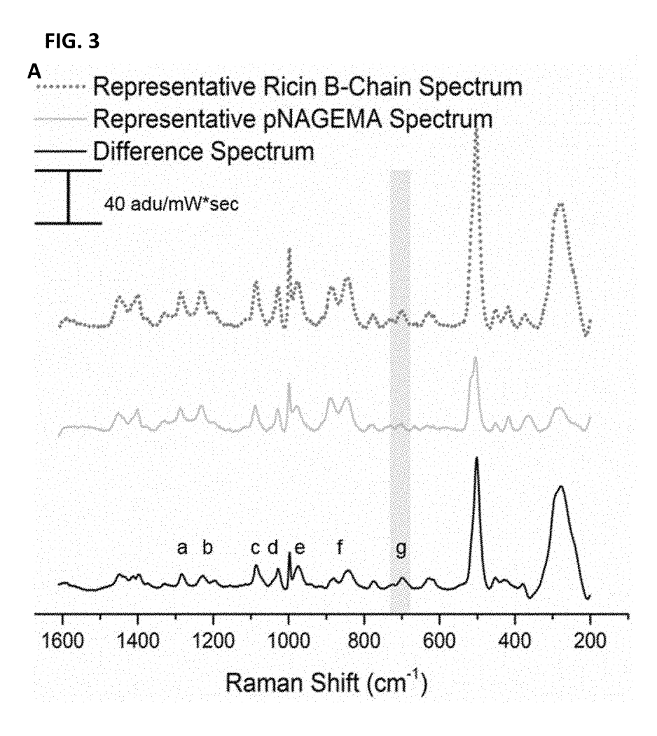

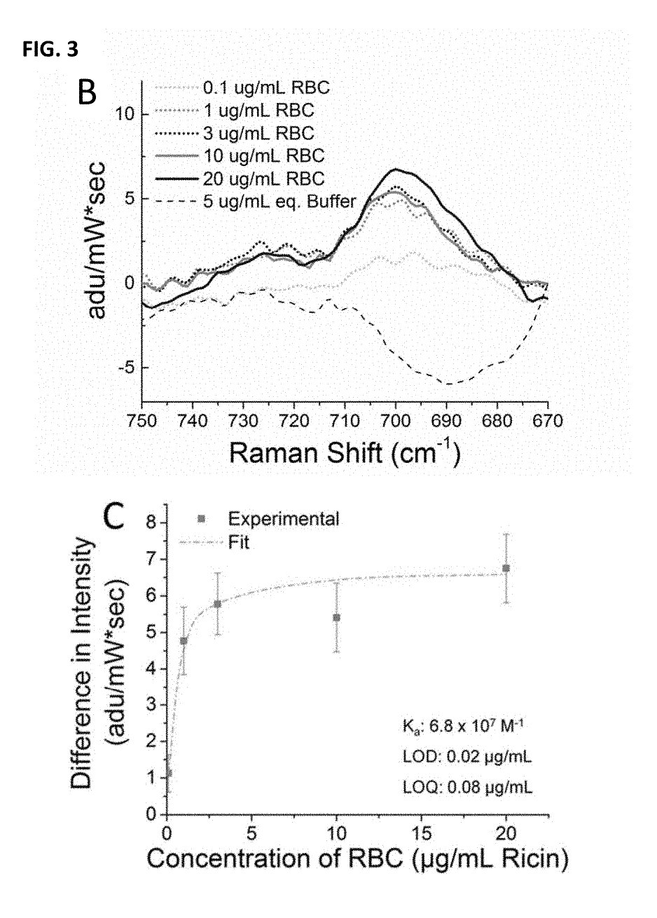

In juice, the buffer and RBC spectra (FIGS. 4B and 4D) contain many of the peaks that were exclusive to RBC in deionized (DI) water (FIG. 3A c, e-g). This can be attributed to the sensor undergoing some non-specific binding with native juice proteins having similar residue vibrations. However, the peak increase at 1280 cm⁻¹ shift, associated with the random coil of the B chain, is still only seen in the RBC-spiked juice experiments and not observed in the control.

Also unique to the RBC difference spectra are the changes seen in the 615-630 cm⁻¹ shift region. Peaks in this range have been previously assigned to RBC's cysteine C-S stretching, but vibrations from 2-mercaptoethanol may be seen in this region (600-660 cm⁻¹ shift).

Both spiked juices show an increased peak intensity at 380 cm⁻¹ shift, indicating a conformational change in chemisorbed polymer. The observed 10 cm⁻¹ shift to higher energy is of polymer peaks that originate from molecule-wide bending, with significant ring distortions through C bending.

Unlike several of the other literature reports of ricin SERS sensors (Table 1), this technique has a quantitative range, and many qualitative spectral changes are preserved in the presence of complex food matrices. Those seen at 1280 cm⁻¹, 615-630 cm⁻¹, and 380 cm⁻¹ shifts agree with past RBC SERS work, allowing for the detection of RBC in fruit juices.

SBA can be detected at a concentration of at least 1 mg/mL, which is a concentration similar to SBA when isolated from soybean oil meal (1.6 mg/g).

### Detection Speed

While the incubation time of six hours presented in the embodiments and examples described herein exceeds the 0.5-1.5 hour detection times of reported SERS ricin sensors, the speed and simplicity of SERS and the high lectin-pNAGEMA Kₐ may facilitate faster detection times.

### Comparison to Other Techniques

In contrast to reported SERS ricin sensing techniques (see Table 1), this disclosure describes using the naturally selective binding of ricin to N-acetyl-galactosamine for SERS detection. Computational modeling can provide a good fit with experimental SERS spectra, enabling the assignment of polymer peaks and insight into lectin-pNAGEMA interactions. Unlike other published ricin SERS sensing schemes, the capture agent presented herein can be directly applied to the sensing of other lectins of interest, such as those relevant to food allergens.

TABLE 1
Comparison of SERS ricin sensing techniques.

| | | | | | | | Sample Prep | | | |
|---|---|---|---|---|---|---|---|---|---|---|
| | | | | | | | and | | | |
| | | Capture | | | | | Detection | | Spectral | Complex |
| SERS Substrate | Target | Agent | LOD | | LOQ | | Time | | Distinction | Matrices |
| Ag dendrites¹ | Ricin B | Aptamer | 10 ng/mL (PBS), | | Non-quantitative | | <40 | min | Spectra, 2nd | Orange |
| | chain | | 50 ng/mL (orange | | | | | | derivative, | juice, milk |
| | | | juice), 100 ng/mL | | | | | | PCA | |
| | | | (milk) | | | | | | | |
| Ag dendrites² | Ricin | Immuno- | 4 | μg/mL (milk) | 4 | μg/mL (milk) | 20 | min | 2nd | Milk |
| | | magnetic | | | | | | | Derivative, | |
| | | separation | | | | | | | PCA | |
| Deposited Ag | Ricin | None | Concentration not | | Not stated | | Not stated | | Spectra | PBS |
| nanoparticles | | | stated | | | | | | | |
| on inverted | | | | | | | | | | |
| pyramidal | | | | | | | | | | |
| nanovoids³ | | | | | | | | | | |
| Ag nanoparticles⁴ | Ricin B | Aptamer | 10.2 | fg/mL | 32 | fg/mL | ~1 | hr | Raman tag of | Orange |
| | chain | | | | | | | | 4,4′-bipyridyl | juice, milk, |
| | | | | | | | | | | blood, urine |
| Au nanoparticles | Ricin | Single- | 8.9 | ng/mL | Not stated | | ~90 | min | Spectra peak | Apple juice, |
| immobilized on | | stranded | | | | | | | ratio | human serum |
| Si wafer, covered | | oligodeoxy- | | | | | | | | |
| in a silver shell | | nucleotides | | | | | | | | |
| post-ricin | | | | | | | | | | |
| attachment⁵ | | | | | | | | | | |
| Gold film over | Ricin B | N-Acetyl- | 20 | ng/mL | 80 | ng/mL | 6 | hr | Difference | Apple Juice, |
| nanospheres | chain | galactosamine | | | | | | | spectra, PCA | orange Juice |
| | | glycopolymer | | | | | | | | |

¹He et al., Chem. Sci., 2011, 2, 1579.
²He et al., J. Food Sci., 2011, 76, N49-N53.
³Wang et al., Nanoscale Res. Lett., 2015, 10.
⁴Zengin et al., J. Mater. Chem. B, 2014, 3, 306-315.
⁵Tang et al., ACS Appl. Mater. Interfaces, 2016, 8, 2449-2455.

This disclosure describes the use of glycopolymers as a novel capture agent for lectins. In particular, the detection of RBC or SBA in this disclosure demonstrates a novel protein sensing scheme, combining the molecular specificity of surface-enhanced Raman spectroscopy (SERS) and the multivalent biological affinity of glycopolymers (polymers with pendant saccharide units). A novel N-acetyl-galactosamine glycopolymer can be synthesized and used in conjunction with film-over-nanospheres (FONs) as a capture layer in the detection of a lectin target via SERS. In one embodiment, a polymer N-acetyl-galactosamine ethyl methacrylamide (pNAGEMA) is used. In one embodiment, a galactose is used.

Synthetic glycopolymers combine numerous benefits, such as biological recognition, increased affinity (due to multivalency), and applicability to multiple lectin targets. Additionally, synthetic methods provide control over polymer chain length, reactive end groups, and the opportunity for further synthetic optimization.

To incorporate the saccharide into a polymer affinity agent, N-acetyl-galactosamine can be synthetically modified at the C1 hydroxyl with a methacrylamido group for reversible addition-fragmentation chain transfer (RAFT) polymerization. A hydrogen bonding-based association may be formed between the C3 and C4 hydroxyls of N-acetyl-galactosamine and the polar residues of RBC's sugar binding site 2. Hydrogen bonding-based associations may be formed between the carbonyl group of the N-acetamide group, as well as the C3, C4, and C6 hydroxyls, of N-acetyl-galactosamine and the polar residues of SBA's binding sites. Hydrophobic interactions between N-acetyl-galactosamine's C1-H, C3-H, and C5-H groups, and the CH₃ of the N-acetamide group with SBA's nonpolar residues may provide further affinity.

RAFT is a popular controlled radical polymerization technique that utilizes a chain transfer agent (CTA) to control the length and dispersity of chains in the polymer mixture. A CTA may be used, such as a trithiocarbonate that is also able to chemisorb to gold, making this a convenient anchoring chemistry for SERS. Any other suitable CTAs that may be used to chemisorb to gold, including as thiols, disulfides, or dithiocarbonates, for example.

Short polymer chain lengths may be targeted to provide enhanced binding via multivalency and ensure that the capture layer does not extend past the enhancing EM field. The polymer length may provide a capture layer that is less than 3 nm thick. In some embodiments, the polymer chains length may range from about 3 to about 10 glycomonomer repeat units. In one example embodiment, a polymer chain length of approximately 10 glycomonomer repeat units is used. In another example embodiment, a polymer chain length of approximately 3 glycomonomer repeat units is used. A non-limiting example of a glycomonomer is N-acetyl-galactosamine ethyl methacrylamide (NAGEMA).

A sensor can include a glycopolymer-functionalized metal substrate, which may be fabricated having pNAGEMA attached to film-over-nanospheres (FONs). In some embodiments, the metal substrate may include a film (e.g., gold) formed over nanospheres (e.g., silica). In some embodiments, the metal substrate may include colloidal gold. A plasmonic metal other than gold can also be used. Non-limiting examples of suitable plasmonic metals include gold, silver, and copper. The metal substrate may then be subjected to a polymer solution including a glycopolymer. The glycopolymers are attached to the substrate (e.g., chemisorbed).

The glycopolymer may include N-acetyl-galactosamine, N-acetyl-glucosamine, glucose, galactose, or mannose. In some embodiments, the glycopolymer may include N-acetyl-galactosamine or galactose. In one embodiment, the glycopolymer may be a polymer N-acetyl-galactosamine ethyl methacrylamide. The glycopolymer can include one or more of a trithiocarbonate, a thiol, a disulfide, and a dithiocarbonate, for example, to facilitate RAFT polymerization.

The sensor can be appropriate for a variety of lectin targets. The glycomonomers that can be incorporated into the glycopolymer of the capture matrix may be versatile. An example of the synthetic versatility of hexose-type monosaccharides is shown in Scheme A. Similar synthetic modification can be applied to any monosaccharide or disaccharide.

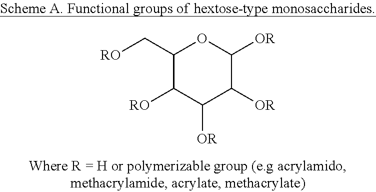

In addition to the N-acetyl-galactosamine methacrylamide usable for RBC and SBA binding, examples of the diverse synthesis of glycomonomers include 6-methacrylamido-6-deoxy trehalose, glucose-6-acrylate-1,2,3,4-tetraacetate, and 2-deoxy-2-methacrylamido glucopyranose.

The synthetic versatility as shown in Scheme A can be used to adjust the synthetic placement of the polymerizable group to maximize binding, which may facilitate developing sugar-lectin sensor pairs for known sugar-lectin interactions (Table A).

TABLE A
Example sugar-lectin interactions that
can be used for developing sensors.

| Sugar | Lectins |
|---|---|
| N-acetyl-galactosamine | Ricin (castor bean), Soybean |
| N-acetyl-glucosamine | Lycopersicon esculentum (tomato), Wheat |
| | Germ, Solanum tuberosum (potato tuber) |
| Glucose | Lens culinaris (lentil seeds), Musa paradisiaca |
| | (banana fruit), Pisum sativum (pea) |
| Galactose | Peanut, Ricin (castor bean), soybean |
| Mannose | Lens culinaris (lentil seeds), Musa paradisiaca |
| | (banana fruit), Pisum sativum (pea) |

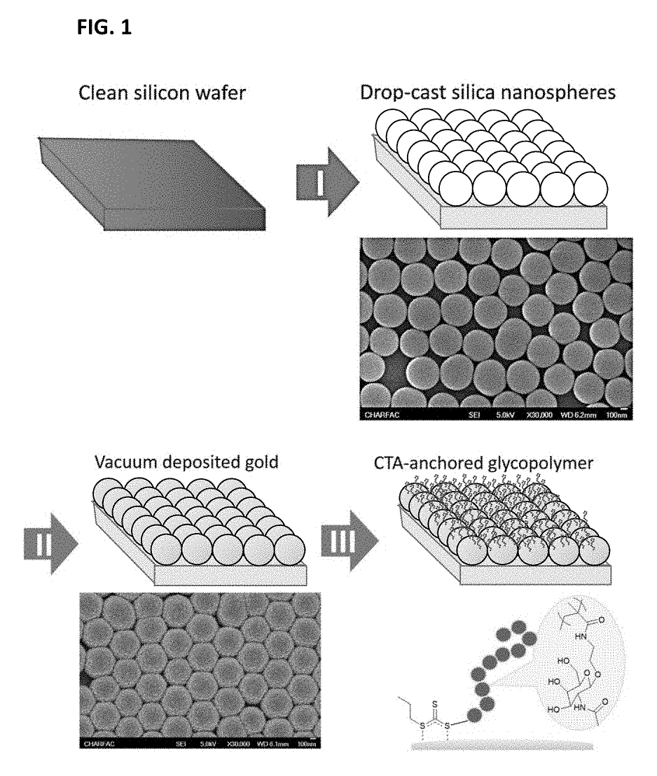

In one embodiment, plasmonic FONs can be submerged in the purified polymer dissolved in ultra-pure water for sensor fabrication. As shown in FIG. 1, (I) silica nanospheres can be drop-coated and self-assembled on a silicon wafer creating a packed sphere mask. (II) gold can be deposited onto the nanosphere template, and substrates with a localized surface plasmon resonance (LSPR) λₘₐₓ in a range, such as between 735-780 nm can be used.

Further, as shown in FIG. 1, (III) the gold (Au) film-over-nanospheres (FONs) may be immersed in a 1 mM polymer solution for a period of time, such as eighteen hours, to allow attachment by chemisorption through the trithiocarbonate of the polymer to the gold. pNAGEMA attachment can be verified by using SERS.

The resulting sensor may include a film-over-nanosphere template and a plurality of glycopolymer chains attached to the film-over-nanosphere template. Each glycopolymer chain may include numerous saccharide moieties for binding a lectin target. For example, the saccharide moieties may include hydroxyl functional groups that interact with amino acids of the lectin target.

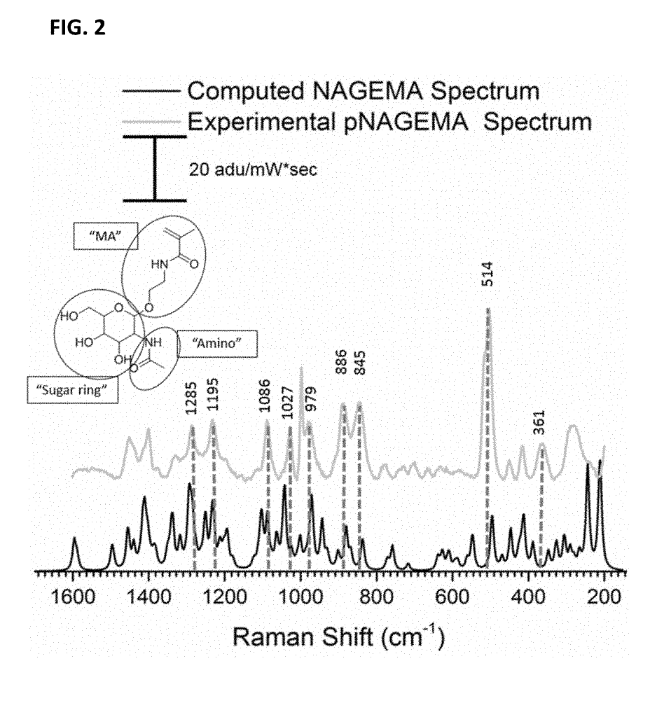

The polymer spectrum may be identified by strong to moderate intensity Raman bands, such as a shift at about 278 cm⁻¹, 361 cm⁻¹, 414 cm⁻¹, 501 cm⁻¹, 514 cm⁻¹, 845 cm⁻¹, 886 cm⁻¹, 979 cm⁻¹, 996 cm⁻¹, 1027 cm⁻¹, 1086 cm⁻¹, 123 cm⁻¹ 1, and/or 1285 cm⁻¹. Examples of possible band assignments are included in Table 2. One representative SERS spectrum for the pNAGEMA attached to the FONs is shown in FIG. 2 (blue curve). The experimental shift and theorized origin of the indicated peaks shown in FIG. 2 are shown in Table 2. The glycopolymer may share a number of peaks with other monosaccharides and polysaccharides such as, for example, galactosamine and chitin. When comparing Raman spectra of monosaccharides and polysaccharides, a broadening of Raman scattering peaks may be observed due to the large number of linked sugars.

TABLE 2
Experimental shift and theorized origin of the indicated peaks.

| Experimental | |
|---|---|
| Raman Shift (cm⁻¹) | Computationally Predicted Source of Vibration |
| 1285 | Sugar ring distortions |
| 1195 | C3—C4 stretching in sugar ring |
| 1086 | C(ring)—N(Amino) stretching and C(ring)—O |
| | (MA) [different linear combinations] |
| 1027 | Molecule-wide vibrations. Sugar ring distortions, |
| | C—O stretching, C—N stretching |
| 979 | Sugar ring distortions coupled to Amino C—N |
| | stretching |
| 886 | MA C—N—C bending |
| 845 | Sugar ring distortions via C—C stretching |
| 514 | Molecule-wide stretching. Sugar ring distortions, |
| | C—OH stretching, O═C—N stretching on MA |
| 361 | Molecule-wide bending, consists of N—C—C in |
| | MA and Amino (coupled symmetrically). |
| | Significant ring distortions through C bending. |

The Raman scattering cross-section for each normal mode may be related to the polarizability derivative with respect to that mode. FIG. 2 shows a computed Lorentzian-broadened spectrum, with a full width half max (FWHM) of 10 cm⁻¹. The FWHM of 10 cm⁻¹ is empirically chosen to make the peaks have widths similar to the examples.

The computational prediction of the NAGEMA spectrum (FIG. 2, black curve) using DFT-based methodologies (Jensen et al., J. Chem. Phys. 2005, 123 (17), 174110) provides a means for Raman peak assignment (Table 3). These assignments can facilitate elucidating how the polymer interacts with the lectin. As shown in FIG. 2, many of the ring distortion peaks, indicated by dashed lines, are shown to change upon incubation with RBC, a result consistent with the N-acetyl-galactosamines confined to the RBC binding pocket.

TABLE 3
Computed spectral peaks with vibration source.

| Raman | |
|---|---|
| Shift | |
| (cm⁻¹) | Source of Vibration |
| 212 | Molecule-wide bending motions that are pronounced in |
| | lengthening the MA chain. Since the backbone is not |
| | modeled, it is expected that this mode maybe shifted |
| | from its true location in the experimental spectra |
| 244 | Molecule-wide bending motions that are pronounced in |
| | lengthening the Amino chain. This mode may not be as |
| | affected by the missing backbone and is only expected |
| | to be shifted a modest amount compared to experiment |
| 306 | Rocking motions in the Amino coupled to significant ring |
| | distortions via C1 and C5 bending |
| 327 | Molecule-wide bending dominated by C—C—N and |
| | C—O—C bending on MA, Amino, and Ring |
| 388 | Molecule-wide bending, consists of N—C—C in MA and |
| | Amino (coupled symmetrically). Significant ring distortions |
| | through C bending |
| 413 | Ring distortions through C—C—C bending and C—C |
| | stretching. There is also bending motion on the MA and |
| | Amino |
| 447 | Umbrella motions on the N in the Amino and MA coupled |
| | to the N—C—C bending |
| 496 | Bending motions on the Amino coupled to ring distortions |
| | via C2 and C5 bending |
| 547 | Molecule-wide stretching. Sugar ring distortions, C—OH |
| | stretching, O═C—N stretching on MA |
| 627 | Sugar ring distortions via C4 bending |
| 758 | C—C stretching on MA |
| 837 | Sugar ring distortions via C—C stretching |
| 880 | MA C—N—C bending |
| 944 | Sugar ring distortions coupled to Amino C—N stretching |
| 970 | C—O stretching in MA, and sugar |
| 1042 | Molecule-wide vibrations. Sugar ring distortions, C—O |
| | stretching, C—N stretching |
| 1087/1102 | C(ring)—N(Amino) stretching and C(ring)—O (MA) |
| | [different linear combinations] |
| 1193 | C3—C4 stretching in sugar ring |
| 1234/1250 | Sugar ring distortions, MA O—C—N bending |
| 1293 | Sugar ring distortions |
| 1316/1337/ | Sugar ring distortions coupled to H—C—H bending on |
| 1338 | MA. Under the same peak CH3 umbrella mode on MA |
| | (1337) and Amino (1338) |
| 1409 | CH bending modes on MA |
| 1455 | CH bending modes on MA |
| 1494/1496 | N—H bending modes on MA and Amino |
| 1589 | C═O stretching on MA. (1596) C═O stretching on Amino |

In one embodiment, sensing can be conducted by incubating a sensor having a glycopolymer-functionalized metal substrate (e.g., including pNAGEMA-modified FONs) in the desired lectin or control solution and then observing the SERS spectrum. A first sensor portion may be incubated in a fluid including a lectin target. A second sensor portion may be incubated in a control fluid including a buffer. The first sensor portion may have a glycopolymer-attached film-over-nanosphere matrix configured to generate a signal-enhancing electromagnetic field in response to incident light.

A first set of data may be generated, via Raman Spectroscopy for example, representing the first sensor portion. A second set of data can be generated, via Raman Spectroscopy for example, representing the second sensor portion.

Sets of spectral data may be generated on the sensor after incubation (e.g., post-incubation data). For example, localized surface plasmon resonance (LSPR) and SERS spectra of the post-incubation FONs may be measured. For conditions acquired in water, a red shift in the LSPR λₘₐₓ can be measured (e.g., using extinction spectroscopy) as shown in FIG. 2.

Sets of spectral data may be generated on the sensor before incubation (e.g., pre-incubation data). Changes in the collected SERS spectra can be subtle until the SERS spectrum of the glycopolymer can be subtracted from the post-lectin incubation spectrum (FIG. 3A). As shown in the Raman difference spectrum, several peak intensities increase. Pre-incubation sets of spectral data may be generated on the first sensor portion and/or second sensor portion.

In some embodiments, one or more sets of spectral data may be predetermined or stored and retrieved. In some embodiments, a first pre-incubation set of spectral data corresponding to a sensor to be subjected to a lectin target and/or a second pre-incubation set of spectral data corresponding to a sensor to be subjected to a buffer may be stored. In some embodiments, the second post-incubation set of spectral data corresponding to the buffer may be determined and recorded for later comparison to a first post-incubation set of spectral data generated from a sample that may contain a lectin target. In some embodiments, a first post-incubation set of spectral data corresponding to a sample having a lectin target may be stored.

Analyzing the difference in spectral data may facilitate identification of lectin-dependent shift regions. For example, a lectin-dependent shift region may be identified where the glycopolymer-bound lectin target vibrates but the glycopolymer, buffer solution, and/or food matrix do not vibrate. Once a shift region is identified, whether the lectin target is present on a substrate may be determined in response to comparing the respective first and second sets of data in the shift region. As shown in FIGS. 3A and 3B, a shift region may be identified at 700 cm⁻¹ for detecting the concentration of ricin B chain bound to a pNAGEMA-functionalized metal substrate in buffer solution.

In some embodiments, the concentration of the lectin target may also be determined in response to the peak intensities in the shift region. For example, a concentration of the lectin target can be determined by analyzing peak intensities in the appropriate shift region in spectral data corresponding to the sensor incubated in solution that may contain the lectin target. The peak intensities in the spectral data may be compared to the spectral data of the sensor pre-incubation and/or the sensor incubated in a buffer or control solution. As shown in FIG. 3C, the peak intensity of the difference between post- and pre-incubation sets of spectral data is proportional to the concentration of the lectin target (e.g., RBC).

In one embodiment, a sensor having a glycopolymer-functional metal substrate is analyzed via Raman Spectroscopy to generate a pre-incubation set of spectral data. The sensor is then incubated in a solution of containing a lectin target and analyzed via Raman Spectroscopy to generate a post-incubation set of spectral data. The pre-incubation set of spectral data is subtracted from the post-incubation set of spectral data to identify peaks that indicate potential Raman shift regions. Another portion of the sensor not exposed to the lectin target is analyzed via Raman Spectroscopy to generate another post-incubation set of spectral data, before incubation and/or after incubation in a buffer solution, to determine whether the buffer solution vibrates in the same shift region. If the glycopolymer and/or buffer solution do not vibrate in a particular shift region, the shift region may be identified as a shift region to monitor for the presence and/or concentration of the lectin target.

The lectin target may also be detected in a complex food matrix (e.g., apple juice or orange juice) using techniques described herein. In some cases, the lectin may be removed from the food matrix once detected or the food matrix may be discarded or otherwise marked as toxic for ingestion.

In some embodiments, the difference between spectral data of the sensor incubated in a lectin target-in-food matrix and spectral data of the sensor incubated in a buffer-in-food matrix may indicate the presence and/or concentration of the lectin target. Possible shift regions that indicate the presence and/or concentration of the lectin target may be identified where the spectrums differ. As shown in FIGS. 4A-D, a shift region of about 1280 cm⁻¹, from about 615 to about 630 cm⁻¹, at about 380 cm⁻¹, or any combination of two or more thereof may be identified for detecting ricin B chain in a food matrix, such as juice (e.g., apple juice, orange juice, etc.).

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

A sensor was fabricated having pNAGEMA attached to FON in Example 1. Sensing experiments were conducted by incubating the sensor having pNAGEMA-modified FONs in a ricin B chain or control solution in Example 2. The strong binding seen between the glycopolymers and the RBC and the real-world risk of contaminated food items prompted investigation of the efficacy of the sensor having pNAGEMA-modified FONs in apple juice with mixing and stagnant orange juice in Example 3.

Throughout the Examples, reference is made to certain characterizations using LSPR and SERS, as described herein.

### LSPR Characterization

The localized surface plasmon resonance (LSPR) was measured from five randomly chosen spots on each FON using an Ocean Optics fiber optic probe.

### SERS Characterization

SERS spectra were recorded from ten spots in a set configuration on each FON using a xyz stage from Thor Labs, and a Snowy Range Instruments SnRI ORS system with 785 nm laser, typically using 3.00-4.00 mW incident power and collection times of 15-30 seconds. The ten SERS spectra collected from each FON were viewed, and those with significantly lower signal-to-noise ratio or a multitude of extraneous peaks were excluded from the FON average spectrum. The FON averaged spectra were then truncated at 1600 cm⁻¹ shift. OriginLab's Origin 9.1 was then used to create and subtract a baseline from each FON average. Briefly, 11 anchor points were found using the first and second derivative and Savitzky-Golay smoothing. The points were connected by B-spline interpolation, utilizing the same number of points as the input spectrum. The spectra were then normalized by the product of the laser power and integration time. Difference spectra were created from the subtraction of a FON's averaged pNAGEMA spectrum from the FON's ultimate condition spectrum.

### Example 1

N-acetyl-galactosamine was synthetically modified at the C1 hydroxyl with an ethyl methacrylamido group for reversible addition-fragmentation chain transfer (RAFT) polymerization (Dhande et al, Biomacromolecules 2016, 17, 830-840). The plasmonic film-over-nanospheres (FONs) were made following a previously published procedure (Kim et al., Analyst 2014, 139 (13), 3227-3234). The FONs were submerged in purified polymer dissolved in ultra-pure water for sensor fabrication.

### Monomer Synthesis

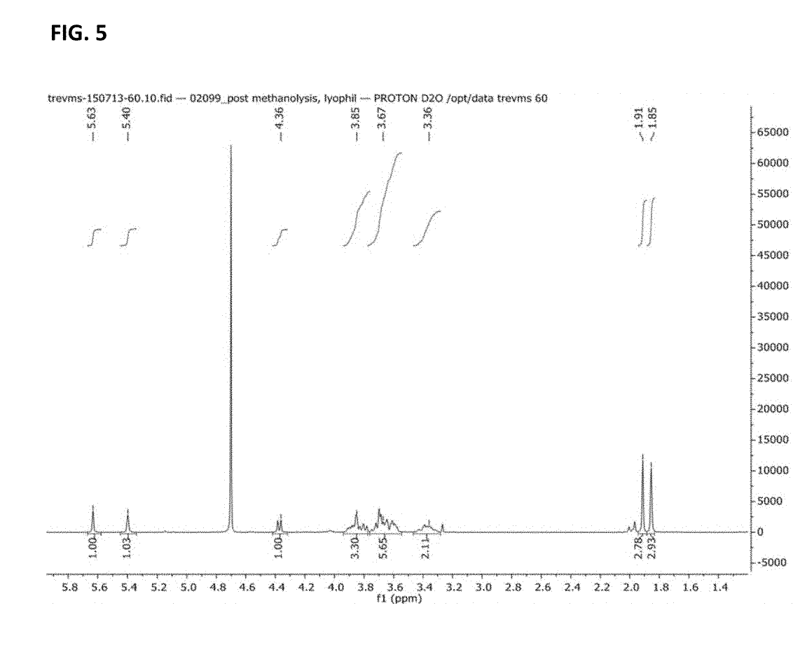

N-acetyl-D-galactosamine (Sigma-Aldrich, St. Louis, Mo.) was synthetically modified at the C1 hydroxyl with an ethyl methacrylamido group as previously described (Dhande et al, Biomacromolecules 2016, 17, 830-840), synthesized from methacryloyl chloride and ethanolamine (Sigma-Aldrich, St. Louis, Mo.), to form a monomer. The monomer synthesis is shown in Scheme 1. Similar yields and purities were achieved. The final product, N-acetyl-galactosamine ethyl methacrylamide (NAGEMA), was used as a powder lyophilized from water. The ¹H NMR of the final, purified NAGEMA is shown in FIG. 5, (400 MHz, deuterium oxide) δ 5.63 (d, J=1.9 Hz, 1H), 5.40 (d, J=2.3 Hz, 1H), 4.37 (d, J=8.5 Hz, 1H), 3.93-3.76 (m, 3H), 3.75-3.56 (m, 6H), 3.46-3.28 (m, 2H), 1.91 (d, J=2.1 Hz, 3H), 1.86 (d, J=1.9 Hz, 3H).

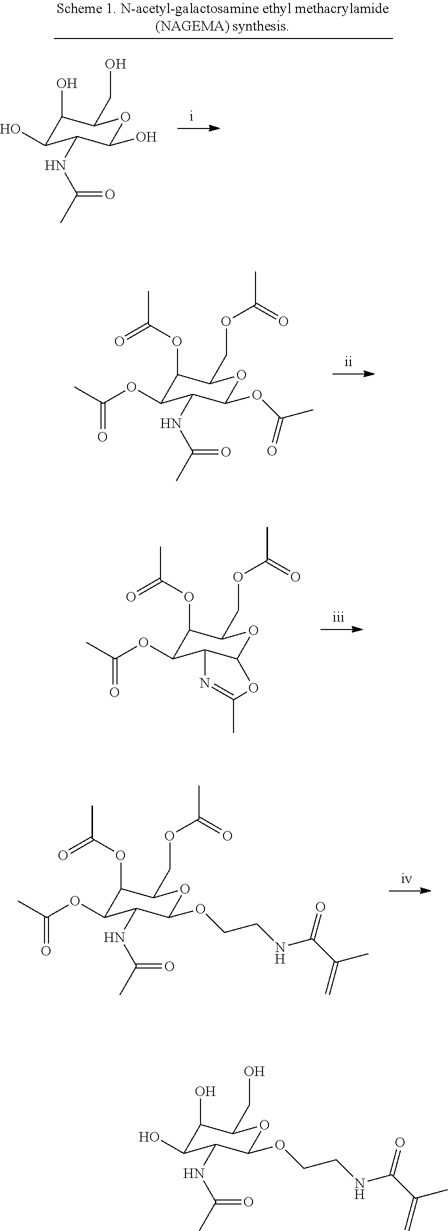 [SMILES: CC(=O)NC1C(O)OC(CO)C(O)C1O ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O ; C=C(C)C(=O)NCCOC1OC(COC(C)=O)C(OC(C)=O)C(OC(C)=O)C1NC(C)=O ; C=C(C)C(=O)NCCOC1OC(CO)C(O)C(O)C1NC(C)=O]

### Glycopolymer Synthesis

Polymerization of the monomer NAGEMA was carried out via reversible addition fragmentation chain transfer (RAFT) polymerization, shown in Scheme 2, utilizing a chain transfer agent (CTA), as previously described (Dhande et al, Biomacromolecules 2016, 17, 830-840). The CTA used was a trithiocarbonate that was also able to chemisorb to gold, making for a convenient anchoring chemistry for SERS (Duwez et al., Macromolecules 2006, 39 (8), 2729-2731; Fustin et al, J. Electron Spectrosc. Relat. Phenom. 2009, 172 (1-3), 104-106). Short polymer chain lengths of approximately 10 N-acetyl-galactosamine ethyl methacrylamide (NAGEMA) repeat units were targeted to provide enhanced binding via multivalency and ensure that the capture layer does not extend past the enhancing EM field.

To carry out all of the experiments, two batches of polymer were synthesized. To decrease reaction times, the polymerization conditions were optimized between the first (used for the experiments in the orange juice matrix) and second synthesis (used for the experiments in deionized water and apple juice) of pNAGEMA for this work. Changes are detailed herein and in Table 4. An average of about 9 to about 10 repeat units and an average molecular number (Mₙ) of about 3.3 to about 3.6 kilodaltons (kDa) was obtained.

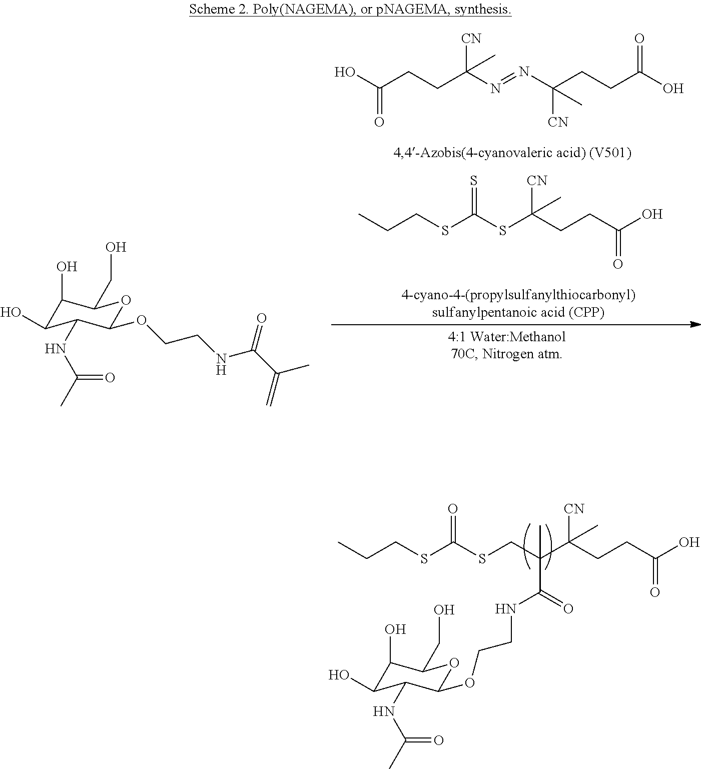

TABLE 4
Polymerization specifics and characterization data

| | | Concen- | | | Average |
|---|---|---|---|---|---|
| | Molar Ratio of | tration of | | | number of |
| | Monomer:CTA:Ini- | Monomer | Mₙ | | Repeat |
| Use | tiator | (M) | (kDa) | [symbol] | Units |
| OJ | 15:1:0.1 | .1 | 3.26 | 1.02 | 9.0 |
| experiments | | | | | |
| DI water | 17:1:0.2 | .5 | 3.55 | 1.05 | 9.9 |
| and AJ | | | | | |
| experiments | | | | | |

For the first reaction, 328 mg (0.988 mmole) NAGEMA, 18.3 mg (0.0659 mmole) 4-cyano-4-(propylsulfanylthiocarbonyl) sulfanylperntanoic acid (CPP, synthesized as previously described (Xu et al., Macromolecules, 2008, 41, 8429-8435)), and 1.85 mg (0.00660 mmole) 4,4′-azobis(4-cyanovaleric acid (V501, >75%; Sigma-Aldrich, St. Louis, Mo.) were dissolved in 9.88 mL 4:1 water/reagent grade methanol. The reaction mixture was stoppered and degassed with nitrogen for 50 minutes. The polymerization was run at 70° C. for 13 hours, with subsequent equimolar additions (0.00660 mmole) of initiator after 5 hours and 10 hours to push the reaction to the desired monomer conversion (60%).

To decrease reaction times, the second synthesis utilized more initiator and an increase in the concentration of the reaction. The reaction flask was charged with 332 mg (1.00 mmole) of monomer dissolved in 1.61 mL of water. To this 0.403 mL reagent grade methanol containing 16.3 mg (0.0588 mmole) of dissolved CPP and 3.30 mg (0.0118 mmole) of dissolved V501 was added. The reaction mixture was stoppered and degassed with nitrogen for 15 minutes. The polymerizations were run at 70° C. for five hours.

Both polymerizations were stopped by the exposure of the reaction mixture to oxygen. Reaction mixtures were dialyzed (MWCO 1000 Da) against MiliQ water overnight, and lyophilized to dryness. The first reaction resulted in 138 mg of polymer (39.9% yield) and the second in 22 mg of polymer (6.3% yield). Polymers were characterized by aqueous (0.1 M Na₂SO₄ in 1.0 v % acidic acid) size exclusion chromatography (SEC) utilizing an Agilent 1260 Infinity Quaternary LC System with Eprogen columns [CATSEC1000 (7 μm, 50×4.6), CATSEC100 (5 μm, 250×4.6), CATSEC300 (5 μm, 250×4.6), and CATSEC1000 (7 μm, 250×4.6)]. The system was equipped with a Wyatt HELEOS II light scattering detector (λ=662 nm), and an Optilab rEX refractometer (λ=658 nm). The Mₙ and [symbol] determined by SEC are listed in Table 1.

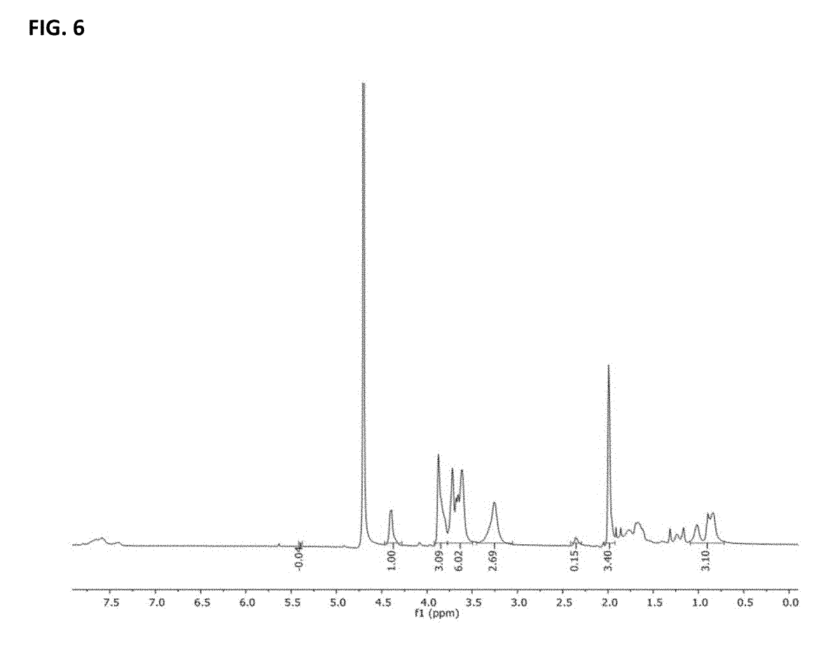

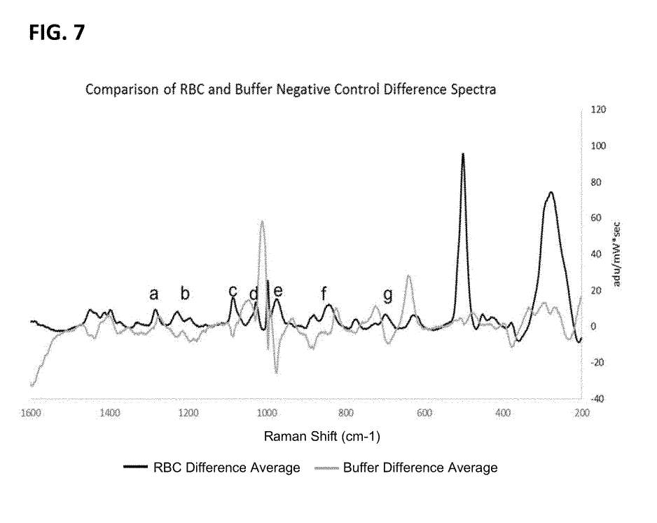

The ¹H NMR of the polymerized NAGEMA is shown in FIG. 6.

### Film-Over-Nanosphere Fabrication

Silica nanospheres having 568-nm-diameter, purchased from Bangs Laboratories (Fishers, Ind.), were refrigerated and resuspended in MilliQ water before use. Self-assembly of the nanosphere mask occurred at room temperature, as previously described (Willets et al., Annu. Rev. Phys. Chem., 2007, 58, 267-297). Briefly, 1.0-2.0 μL of 5% nanosphere suspension in water were placed onto a piranha-cleaned silicon wafer. The wafer was agitated by hand until the nanosphere suspension was evenly distributed, then the solution was allowed to dry in ambient conditions.

The gold (99.999% Au) was purchased from Kurt J Lesker (Jefferson Hills, Pa.) and was deposited to a thickness of 80 nm using a Denton Vacuum deposition chamber (with quartz crystal microbalance) at a rate of 1.2-2.0 angstroms/min at a base pressure of 10⁻⁵ Torr.

### Sensor Fabrication

Fabrication of the sensor is illustrated in FIG. 1. Silica nanospheres, were drop-coated and self-assembled on a silicon wafer creating a packed sphere mask. (II) 80 nm of gold was deposited onto the nanosphere template, and substrates with a localized surface plasmon resonance (LSPR) λₘₐₓ between 735-780 nm were utilized. (III) The Au FONs were immersed in a 1 mM polymer solution for eighteen hours to allow attachment by chemisorption through the trithiocarbonate of the polymer to the gold.

The pNAGEMA attachment was verified using SERS. The polymer spectrum was identified by strong to moderate intensity Raman bands at shifts of 278 cm⁻¹, 361 cm⁻¹, 414 cm⁻¹, 501 cm⁻¹, 514 cm⁻¹, 845 cm⁻¹, 886 cm⁻¹, 979 cm⁻¹, 996 cm⁻¹, 1027 cm⁻¹, 1086 cm⁻¹, 1231 cm⁻¹, and 1285 cm⁻¹.

A representative SERS experimental spectrum for the pNAGEMA attached to the FONs is shown in FIG. 2, as well as a computer NAGEMA spectrum to facilitate identifying possible band assignments. Possible band assignments are shown in Table 5.

The calculations of the normal Raman scattering of the monomer unit, NAGEMA, were performed using TDDFT implemented in the ADF (Amsterdam Density Functional) program (te Velde et al., J. Comput. Chem., 2001, 22, 931-967; Guerra et al., Theor. Chem. Acc., 1998, 99, 391-403; Baerends et al., ADF2014, 2014). The AOResponse module (Jensen et al., J. Chem. Phys., 2005, 123, 174110) was used to calculate the polarizability derivatives using numerical displacements of the molecular coordinates for each normal mode. The BP86 exchange-correlation (XC) functional (Becke, A D, Phys. Rev. A, 1988, 38, 3098-3100; Perdew, J P, Phys. Rev. B, 1986, 33, 8822-8824) and a triple-ζ polarized Slater type (TZP) basis set were used. The BP86 XC function was chosen because it is reasonably accurate (within 20-30 cm⁻¹) in reproducing the frequencies of the vibrational modes (Dhande et al, Biomacromolecules 2016, 17, 830-840). At all steps, the COSMO-RS implicit solvent parameters for water were used to approximate the effects of an aqueous environment (Pye et al., Can. J. Chem., 2009, 87, 790-797)

### Example 2

### Method

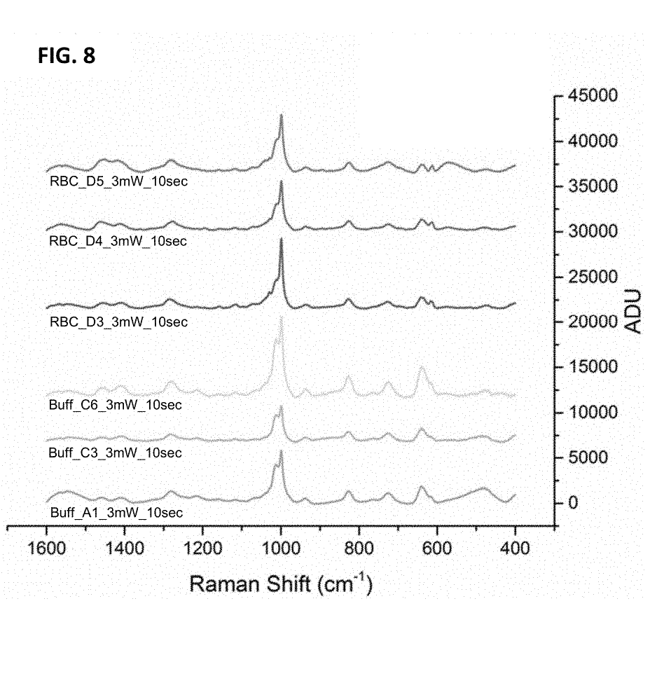

Sensing experiments were conducted by incubating pNAGEMA-modified FONs in an RBC purchased from Vector Laboratories (Burlingame, Calif.) or control solution for six hours, with constant mixing via an orbital shaker. Corresponding bare FON controls are shown in FIG. 8. Control solutions contained 2-mercaptoethanol (Sigma-Aldrich, St. Louis, Mo.) at the same concentration as present in the RBC solutions to prevent the formation of disulfide bonds between RBC proteins. Post-incubation, the FONs were rinsed with deionized water and dried under nitrogen.

Post-incubation, the FONs were rinsed with deionized water and dried under nitrogen. The localized surface plasmon resonance (LSPR) and SERS spectra were then measured using extinction spectroscopy.

### Results & Discussion

For all conditions acquired in water, a red-shift in the LSPR λₘₐₓ was measured using extinction spectroscopy as shown in Table 5, which may be a change due to the increased refractive index of adsorbed species on the sensor surface.

TABLE 5
LSPR shifts due to condition incubation.

| | Average LSPR |
|---|---|
| FONs | λₘₐₓ shift (nm) |
| Incubated w/0.1 μg/mL RBC | 8 ± 2.9 |
| Incubated w/1 μg/mL RBC | 4 ± 2.6 |
| Incubated w/3 μg/mL RBC | 3 ± 1.9 |
| Incubated w/10 μg/mL RBC | 0 ± 1.0 |
| Incubated w/20 μg/mL RBC | 8.0 ± 0.6 |
| Incubated w/equivalent of 5 μg/mL Buffer | 3.0 ± 0.2 |
| Incubated w/3 μg/mL RBC in AJ | −12 ± 7.2 |
| Incubated w/equivalent of 3 μg/mL Buffer in AJ | −24 ± 20 |
| Incubated w/3 μg/mL RBC in OJ | −20 ± 7.4 |
| Incubated w/equivalent of 3 μg/mL RBC in OJ | −3 ± 2.1 |

Changes in the collected SERS spectra are subtle until the SERS spectrum of the glycopolymer is subtracted from the post-RBC incubation spectrum (FIG. 3A). As shown in the Raman difference spectrum, several peak intensities increase. The spectral changes that are independent of the negative control are labeled (a-g). The negative control of baseline corrected SERS spectra of bare FONs are shown for comparison in FIG. 8.

Using previously measured Raman spectra of RBC and its cyclic amino acids (which have the largest scattering cross-section), the likely molecular origin of the spectral changes was discerned. The signal at 1280 cm⁻¹ shift (a) was assigned to the unstructured coil of RBC, and the signal at 1088 cm⁻¹ shift (c) was attributed to C—C and C—N stretching vibrations within the protein, which overlays similar C (ring)-N (amino) and C (ring)-O (methacrylamide) stretching in the polymer.

While these signals may be typical in Raman analysis of any random coil proteins, in this disclosure, the co-location with polymer peaks makes them qualitative indicators of RBC. The increases at 990 cm⁻¹ shift (d) and 1024 cm⁻¹ shift (e) are from phenylalanine and overlap with sugar ring distortions and C—O stretches. Vibrations from tyrosine and tryptophan contribute to the increases seen between 830-880 cm⁻¹ shift (f) and that at 1190 cm⁻¹ shift (b).

In both polymer and protein, the 830 cm⁻¹ shift region is occupied by peaks due to ring distortions. In this region, the overlap of these makes quantitative analysis of band intensity difficult.

The addition and orientation of RBC, as well as the resulting polymer displacement, lead to complicated peak intensity changes. Highlighted in FIG. 3A and FIG. 3B, is an emerging peak in the 700 cm⁻¹ shift region (g). Literature precedent attributes this peak to tyrosine and tryptophan vibrations.

While all the previously mentioned peak intensity increases can be attributed to RBC features, the 700 cm⁻¹ shift region (g) is unobscured by vibrations from the polymer, making it the optimal portion of the spectrum for quantification. The peak appears to be concentration dependent (FIG. 3C) and is clearly not a result of the 2-mercaptoethanol present in the negative control RBC buffer (FIG. 3B).

Using the difference due to incubation with RBC, the limit of detection (LOD) and limit of quantification (LOQ) are calculated to be 0.02 μg/mL RBC and 0.08 μg/mL RBC, respectively. The limit of detection (LOD) was calculated on spectral difference data using the procedure published in Clinical and Laboratory Standards Institute (CLSI) guideline EP17 (Armbruster et al., Clin. Biochem. Rev., 2008, 29, S49-S52). The limit of quantitation (LOQ) was calculated as 3.3×LOD.

Therefore, detection can occur well below the 6 μg/mL estimated to be dangerous to an adult by oral ingestion over 24 hours of exposure. Plotting the increased amplitude of this peak against RBC concentration yields a Langmuir type plot that is nonlinear past 1.0 μg/mL RBC.

At higher levels, the sensor may be utilized for simple “yes/no” detection. The shape of the plot indicates that at higher concentrations, the sensor is saturated. By fitting this plot to the Langmuir equation (Equation 1), an association constant (Kₐ) for the lectin-saccharide interaction was calculated. The association constant Kₐ may be described as an apparent association constant. To calculate the Kₐ, the data was fit to the Langmuir-type isotherm based on signal differences according to Equation 1:

$${θ}_{rd} • \frac{{K}_{a} M}{1 + {K}_{a} M}$$

Kₐ was computed from Microsoft Excel solver by minimizing the absolute value of the sum of the difference of the computed and collected signal differences.

The derived Kₐ of 6.8×10⁷ M⁻¹ suggests strong, specific binding. For comparison, results obtained in the literature from surface plasmon imaging studies on the lectin jacalin and surface bound galactose resulted in a Kₐ of 2.2×10⁷ M⁻¹.

### Example 3

### Method

The juices purchased from Simply Orange Juice Company (Apopka, Fla.) were spiked with the RBC or the equivalent concentration of buffer, and each condition was performed in triplicate. For the apple juice and stagnant orange juice, RBC concentrations relevant to oral exposure (3 μg/mL and 10 μg/mL, respectively) were introduced.

Initial data analysis of RBC exposures in juice utilized principle component analysis (PCA) of the raw spectra. PCA is a statistical procedure that dimensionally reduces a data set with a large number of variables (such as spectral points across multiple conditions) and can be applied in a qualitative manner to visualize variance and outliers, even in complex spectra.

Principle component analysis (PCA) was performed using scripts. The PCA and subroutines scripts were used as received on CANOPY (Enthought, Inc., Austin, Tex.). The SFP_SERS_barcode script was only modified to adjust the range of the spectra analyzed (Parameters, min_cm: 200, max_cm: 1500), and the parameter ‘variance_to_explain’ set at 0.999. All raw individual spectra that were to be included in the FON averaged spectra were imported into the provided ‘data’ file, and assigned a color code specified in the SFP_SERS barcode script. The output graphs were used as qualitative indicators of the degree to which incubation condition (indicated by the assigned colors) correlated to the variance observed between raw spectra.

LSPR and SERS were measured. Because the SERS signal measured from the FON substrates post-juice incubation was so low, all juice data were normalized by the maximum of the pNAGEMA spectrum to generate meaningful difference spectra.

### Results & Discussion

The LSPR of the FON substrate in all juice conditions was blue-shifted relative to the freshly prepared samples. It is likely that the blue-shift is due to the juice causing some changes in surface roughness, effectively decreasing the nanostructured roughness aspect ratio.

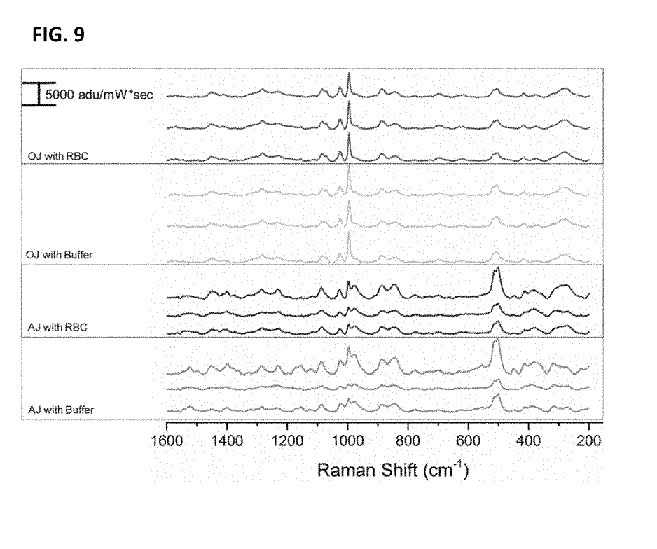

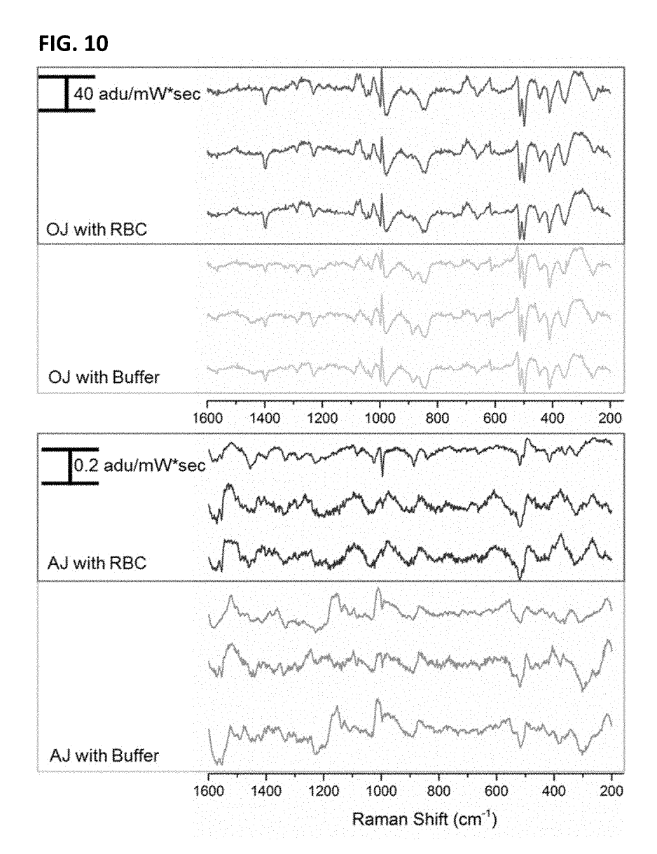

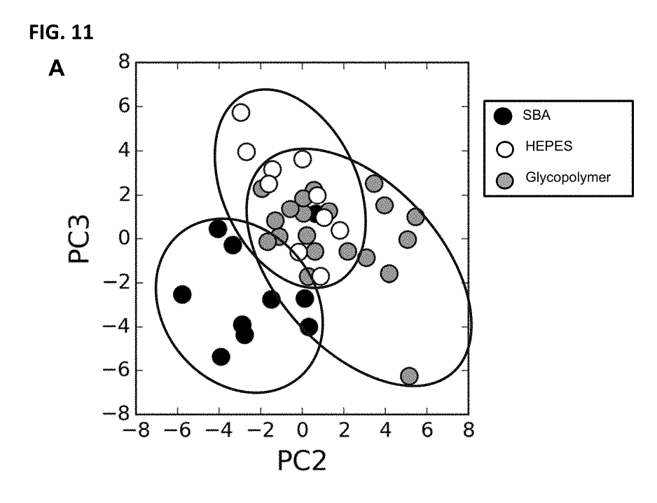

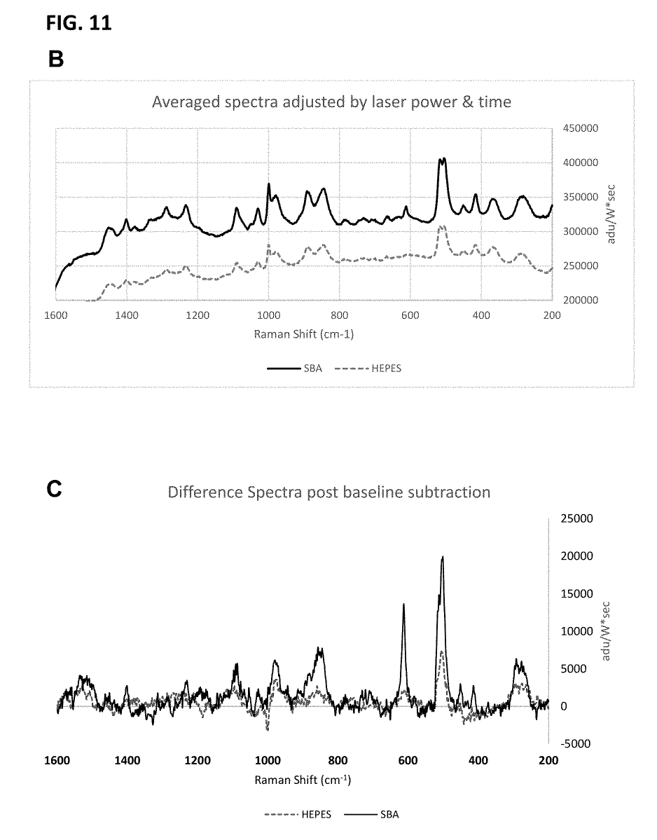

As seen in FIG. 4, the PCA of the control and spiked juice conditions (FIGS. 4A and 4C) reveal that there is variance in the spectra that correlates with the presence of RBC. The individual FON substrate spectra can be seen in FIG. 9 and FIG. 10.

In juice, the buffer, as well as the RBC, spectra (FIGS. 4B and 4D) contain many of the peaks that were exclusive to RBC in deionized water (FIG. 3A c, e-g). This can be attributed to the sensor undergoing some non-specific binding with native juice proteins having similar residue vibrations. However, the peak increase at 1280 cm⁻¹ shift, associated with the random coil of the B chain, is still only seen in the RBC-spiked juice experiments and not observed in the control.

Also unique to the RBC difference spectra are the changes seen in the 615-630 cm⁻¹ shift region. Peaks in this range have been previously assigned to RBC's cysteine C—S stretching, but vibrations from 2-mercaptoethanol may be seen in this region (600-660 cm⁻¹ shift).

Both spiked juices demonstrate an increase peak intensity at 380 cm⁻¹ shift indicating a conformational change in chemisorbed polymer. The observed 10 cm⁻¹ shift to higher energy is of polymer peaks that originate from molecule-wide bending, with significant ring distortions through C bending.

### Example 4

### Method

Using techniques described in this disclosure, a polymer was formed that is shorter than than the one used for ricin B-chain (RBC) was synthesized. pNAGEMA₃ was formed, which had an average number of repeat units was 3 as opposed to 9. The pNAGEMA₃ was bound to a FON substrate. A 10 mM solution of 4-(2-hydroxyethyl)-1-piperazineethanesulfonic acid (HEPES) was used as a control buffer (Sigma-Aldrich, St. Louis, Mo., >99.5%), with 0.1 mM Ca²⁺. Raman shift spectra were captured for FON substrates with only pNAGEMA₃, a sensor incubated in HEPES buffer, and a sensor incubated in SBA. The PCA, averaged spectra, and difference spectra were calculated.

### Results & Discussion

The PCA visually shows a difference in variance among spectra of the pNAGEMA, HEPES buffer incubated, and SBA incubated sensors. The spectrum from the SBA incubated sensor exhibits a new peak at 600 cm⁻¹ and several other features not seen in the HEPES buffer incubated sensor. The shorter galactose-based glycopolymer (pNAGEMA₃) was shown to bind to the SERS substrate and was shown to have a detectable an affinity for SBA.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of making a sensor, the method comprising:
subjecting a metal substrate to a solution comprising at least one glycopolymer chain configured to bind to a lectin target; and
attaching the at least one glycopolymer chain to the metal substrate to form a glycopolymer-functionalized metal substrate configured to bind to a lectin target.

2. The method according to claim 1, wherein the at least one glycopolymer chain comprises at least one of N-acetyl-galactosamine, N-acetyl-glucosamine, glucose, galactose, and mannose.

3. The method according to claim 1, wherein the at least one glycopolymer chain comprises a repeat unit of N-acetyl-galactosamine ethyl methacrylamide.

4. The method according to claim 1, wherein the at least one glycopolymer chain comprises at least one of a trithiocarbonate, a thiol, a disulfide, and a dithiocarbonate.

5. The method according to claim 1, wherein attaching the at least one glycopolymer chain to the substrate comprises chemisorption of the at least one glycopolymer chain to the metal substrate.

6. The method according to claim 1, further comprising forming the metal substrate with a plasmonic metal comprising at least one of gold, copper, and silver.

7. A method of using a sensor, the method comprising:
incubating a first sensor portion in a sample fluid and a second sensor portion in a control fluid, the first and second sensor portions each comprising a glycopolymer-functionalized metal substrate configured to generate a signal-enhancing electromagnetic field in response to incident light;
generating, via Raman Spectroscopy, after incubating the first and second sensor portions, a first post-incubation set of spectral data representing the first sensor portion and a second post-incubation set of spectral data representing the second sensor portion; and
determining whether a lectin target is present in the sample fluid in response to comparing the first and second post-incubation sets of spectral data in a shift region, the shift region being dependent on a concentration of the lectin target in the sample fluid.

8. The method according to claim 7, further comprising:
generating, via Raman Spectroscopy, before incubating the first and second sensor portion, a pre-incubation set of spectral data representing the first and second sensor portions; and
determining a potential shift region in response to comparing the pre-incubation set of spectral data to at least one of the first and second post-incubation sets of spectral data.

9. The method according to claim 7, wherein the shift region is defined at about 700 cm⁻¹.

10. The method according to claim 7, wherein the shift region is defined at about 1280 cm⁻¹, from about 615 cm⁻¹ to about 630 cm⁻¹, at about 380 cm⁻¹, or any combination of two or more thereof.

11. The method according to claim 7, wherein the shift region is defined at about 600 cm⁻¹.

12. The method according to claim 7, further comprising determining a concentration of the lectin target in the sample fluid in response to comparing peak intensities in the shift region of the first and second sets of spectral data.

13. A sensor comprising:
a metal substrate comprising a plasmonic metal; and
at least one glycopolymer chain attached to the metal substrate configured to bind to a lectin target.

14. The sensor according to claim 13, wherein the at least one glycopolymer chain comprises at least one of N-acetyl-galactosamine, N-acetyl-glucosamine, glucose, galactose, and mannose.

15. The sensor according to claim 13, wherein the at least one glycopolymer chain comprises a repeat unit of N-acetyl-galactosamine ethyl methacrylamide.

16. The sensor according to claim 13, wherein the metal substrate comprises at least one of gold, copper, and silver.

17. The sensor according to claim 13, wherein the metal substrate comprises one of a film of gold over silica nanosphere matrix and a colloidal gold substrate.

18. The sensor according to claim 13, wherein the at least one glycopolymer chain is configured to bind to at least one of a lectin food allergen and a lectin toxin.

19. The sensor according to claim 13, wherein the at least one glycopolymer chain is configured to bind to a ricin B chain.

20. The sensor according to claim 13, wherein the at least one glycopolymer chain is configured to bind to a soybean agglutinin.
